(19) United States
(12) Patent Application Publication
(10) Pub. No.: US 2005/0118199 A1
(43) Pub. Date: Jun. 2, 2005

(54) Process for covalently conjugating polysaccharides to microspheres or biomolecules

(76) Inventors: Mark T. Esser, Collegeville, PA (US); Sonela A. Schlottmann, Newbury Park, CA (US)

Correspondence Address: MERCK AND CO., INC, P O BOX 2000, RAHWAY, NJ, 07065-0907, US

(21) Appl. No.: 10/960,522

(22) Filed: Oct. 7, 2004

Related U.S. Application Data
(60) Provisional application No. 60/509,189, filed on Oct. 7, 2003.

(51) Int. Cl. A61K 39/385; A61K 39/09; C07K 14/195; C08B 37/00

(52) U.S. Cl. 424/244.1; 424/490; 536/53; 536/55.3; 530/395

(57) ABSTRACT

The present invention relates generally to novel processes for covalently conjugating polysaccharides to microspheres or other biomolecules, and more specifically to the use of 4-(4,6-dimethoxy[1,3,5]triazin-2-yl)-4-methyl-morpholinium chloride (DMTMM) in said processes

### CROSS-REFERENCE TO RELATED APPLICATIONS

The present invention is related to U.S. provisional application Ser. No. 60/509,189, filed Oct. 7, 2003, the contents of which are hereby incorporated by reference.

### FIELD OF THE INVENTION

The present invention relates to methods for conjugating polysaccharides to microspheres or biomolecules. Such methods are highly valuable in the construction of reliable assays for the detection of an antibody corresponding to the polysaccharide antigen. The present invention also relates to the use of 4-(4,6-dimethoxy[1,3,5]triazin-2-yl)-4-methyl-morpholinium chloride (DMTMM) in said methods.

### BACKGROUND OF THE INVENTION

Polysaccharides (PSs) are a broad family of polymeric molecules found in a variety of organisms. For example, the capsule and cell walls of bacteria and fungi are essentially comprised of PSs composed of specific repeat units. These capsular polysaccharides bear epitope motifs that are usually not found in mammals and can therefore mediate immunogenicity. Such polysaccharides are therefore useful for the preparation of vaccines against bacterial diseases such as meningitis, pneumonia, and typhoid fever. Moreover, immunoassays for the detection of antibodies corresponding to polysaccharide antigens may be used to diagnose infectious diseases and to assess the safety and efficacy of polysaccharide vaccines.

While various immunoassay techniques are well-known in the art for detecting antibodies corresponding to PS antigens (e.g., radioimmunoassays (RIAs), enzyme-linked immunosorbent assays (ELISAs), Western blotting, immunofluorescent assays, etc.), the most common immunoassays include a solid phase matrix to which PSs are bound. Indeed, the immobilization of PSs to the solid phase is usually one of the first steps in preparing an immunoassay. The PSs may be bound to solid supports through a non-covalent chemical bond (e.g., through attachment by van der Waals forces, hydrophobic interdigitation, ionic bonding, etc.) or covalently, i.e., through sharing of valence electrons between an atom on the solid surface and an atom on the PS.

Non-covalent immobilization of PSs onto solid surfaces (coating) is generally time, reagent, and labor consuming because the optimal coating conditions vary among PSs from different bacteria strains as well as between serotypes of the same bacteria. This variability is often not acceptable because there is a significant impact on the accuracy and reproducibility of quantitative determinations. For the same reasons, a simultaneous immobilization of two or more different PSs onto the same surface is often very difficult. In addition, the potential tendency of PSs to form micelles (or aggregates) can lead to decreased and unpredictable coating stability and reduce the long-term stability of the coating. Often various micelle-dispersing agents (detergents) must be added to the coating solution, thereby introducing additional assay variability.

Although techniques for covalent attachments of PSs to solid surfaces overcome some of these problems, they suffer from many others. Current methods of covalent attachment of PSs to solid support are generally limited to PS modification followed by reaction with appropriately functionalized solid supports. For example, PSs may be oxidized, followed by 1-ethyl-3-[3-dimethylaminopropyl]carbodiimide hydrochloride (EDC)-mediated conjugation to microspheres containing hydrazide moieties (Schlottman et al., Oak Ridge Conference, May 2000), or they may be functionalized, e.g., with poly-(1)-lysine and cyanuric chloride, followed by EDC-mediated attachment of the modified PSs to carboxyl containing beads (Pickering et al., Am. J. Clin. Pathol., 2002, 117, 589-596). However, the PS oxidation method affects the epitopes of the PSs which may reduce their antigenicity, immunogenicity, and specificity in an assay, and the poly-(1)-lysine/cyanuric chloride chemistry is not well reproducible.

The above-described limitations for the detection of antibodies corresponding to PS antigens have been overcome with the methods of the present invention which allow for reliable, consistent, and immunospecific attachment of various types of PSs to solid supports and biomolecules.

### SUMMARY OF THE INVENTION

This invention provides a method for coupling a polysaccharide to a microsphere or a biomolecule comprising activating said polysaccharide with 4-(4,6-dimethoxy[1,3,5]triazin-2-yl)-4-methyl-morpholinium chloride and subsequently reacting the activated polysaccharide with said microsphere or biomolecule.

This invention further provides a method for coupling a polysaccharide to a microsphere or a biomolecule comprising activating said microsphere or biomolecule with 4-(4,6-dimethoxy[1,3,5]triazin-2-yl)-4-methyl-morpholinium chloride and subsequently reacting the activated microsphere or biomolecule with said polysaccharide.

In other embodiments of the present invention, a method is provided for assaying for an anti-polysaccharide antibody comprising contacting a sample containing said anti-polysaccharide antibody with a microsphere-polysaccharide conjugate prepared by the methods of the present invention, and measuring the amount of any anti-polysaccharide antibody bound to said microsphere-polysaccharide conjugate.

In certain embodiments of the present invention, a method is provided for detecting a disease, disorder or condition where anti-polysaccharide antibody levels are altered comprising contacting a sample of bodily tissue or fluid with a microsphere-polysaccharide conjugate prepared by the methods of the present invention, wherein said anti-polysaccharide antibody binds to said microsphere-polysaccharide conjugate, and measuring the amount of any anti-polysaccharide antibody bound to said microsphere-polysaccharide conjugate, wherein the amount of said anti-polysaccharide antibody is diagnostic for said disease, disorder or condition.

Another aspect of the present invention provides for a method for assessing the efficacy of a vaccine which vaccine alters anti-polysaccharide antibody levels in a mammal comprising administering an effective amount of said vaccine to said mammal; allowing said mammal to develop anti-polysaccharide antibodies; contacting a sample of bodily tissue or fluid from said mammal with a microsphere-polysaccharide conjugate prepared by the method of the present invention, wherein said anti-polysaccharide antibody binds to said microsphere-polysaccharide conjugate; and measuring the amount of any anti-polysaccharide antibody bound to said microsphere-polysaccharide conjugate, wherein the amount of said anti-polysaccharide antibody is diagnostic for the efficacy of said vaccine.

Furthermore, this invention provides a new method of conjugating polysaccharides to biomolecules such as protein carriers (e.g., Outer Membrane Protein Complex of N. Meningitidis, or OMPC) in order to create potential vaccine candidates.

It is yet another aspect of the present invention to provide a microsphere-polysaccharide or biomolecule-polysaccharide conjugate prepared by methods described herein.

Additional embodiments will be evident from the following detailed description.

### BRIEF DESCRIPTION OF THE DRAWINGS

FIG. 1 is a bar graph showing the comparison of raw median fluorescence intensities (MFIs) for serotypes 4, 5, 6B, 12F, 19F, and 23F of pneumococcal polysaccharides coupled to xMAP microspheres using either poly-(1)-lysine (PLL) or DMTMM coupling chemistries in order to assess potential advantages of ADH-DMTMM coupling method over the published poly-(1)-lysine methodology. Six serotypes with poor results using the poly-(1)-lysine method were coupled using the ADH-DMTMM chemistry.

FIG. 2 is a bar graph showing the comparison of raw median fluorescence intensities for native and DMTMM-activated PnPS 14, a neutral polysaccharide, attached to xMAP microspheres with or without an adipic acid dihydrazide linker:
a) PnPS 14 was activated with DMTMM and conjugated to carboxylate beads (COOH-DMTMM);
b) PnPS 14 was not activated with DMTMM before adding to carboxylate beads (adsorption only, COOH-Native);
c) PnPS 14 was activated with DMTMM and conjugated to carboxylate beads modified with adipic acid dihydrazide (ADH-DMTMM);
d) PnPS 14 was not activated with DMTMM before adding to carboxylate beads modified with adipic acid dihydrazide (ADH-Native).

FIG. 3 is a bar graph showing the comparison of raw median fluorescence intensities for native and DMTMM-activated PnPS 18C, a negatively charged phosphate-containing polysaccharide, attached to xMAP microspheres with or without adipic acid dihydrazide linker:
a) PnPS 18C was activated with DMTMM and conjugated to carboxylate beads (COOH-DMTMM)
b) PNPS 18C was not activated with DMTMM before adding to carboxylate beads (adsorption only, COOH-Native)
c) PnPS 18C was activated with DMTMM and conjugated to carboxylate beads modified with adipic acid dihydrazide (ADH-DMTMM)
d) PnPS 18C was not activated with DMTMM before adding to carboxylate beads modified with adipic acid dihydrazide (ADH-Native)

FIG. 4 is a graph that shows the standard curves produced using a standard reference serum (NJSS) on a multiplexed assay for 12 serotypes.

FIG. 5 is a graph demonstrating the reproducibility of the COOH-DMTMM chemistry for serotype 6B using PnPS-microspheres prepared by three different analysts.

### DETAILED DESCRIPTION OF THE INVENTION

In one embodiment, this invention provides a method for coupling a polysaccharide to a microsphere or a biomolecule comprising activating said polysaccharide with 4-(4,6-dimethoxy[1,3,5]triazin-2-yl)-4-methyl-morpholinium chloride (DMTMM) and subsequently reacting the activated polysaccharide with said microsphere or biomolecule. In a class of this embodiment the coupling provides for a covalent attachment of a polysaccharide to a microsphere or a biomolecule.

In another embodiment, this invention provides a method for coupling a polysaccharide to a microsphere or a biomolecule comprising activating said microsphere or said biomolecule with 4-(4,6-dimethoxy[1,3,5]triazin-2-yl)-4-methyl-morpholinium chloride and subsequently reacting the activated microsphere or the activated biomolecule with said polysaccharide. In a class of this embodiment the coupling provides for a covalent attachment of a polysaccharide to a microsphere or a biomolecule.

In certain embodiments of the present invention, the polysaccharide may be immunogenic and/or antigenic. Preferably, the method for covalently coupling a polysaccharide to a microsphere or a biomolecule does not change the immunogenicity and/or the antigenicity of the polysaccharide.

In certain embodiments of the present invention, the polysaccharide is a bacterial polysaccharide. In one class of this embodiment the bacterial polysaccharide is isolated from bacteria selected from the group consisting of Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, Hemophilus influenzae, Escherichia coli, Shigella spp., Erlichia spp., and Rickettsia spp and from fungi such as Candida albicans, Candida kefyr, Cryptococcus neoformans, Hansenula anomala, and Hansenula arabitolgens. In another class of this embodiment, the polysaccharide is a capsular polysaccharide isolated from Streptococcus pneumoniae. In a subclass of this class, the polysaccharide is of a serotype selected from the group consisting of 1, 2, 3, 4, 5, 6B, 7F, 8, 9N, 9V, 10A, 11A, 12F, 14, 15B, 17F, 18C, 19A, 19F, 20, 22F, 23F, and 33F. In another class of this embodiment the bacterial polysaccharide is prepared synthetically.

In certain embodiments, the microsphere for conjugation to a polysaccharide is a polymer selected from the group consisting of a polystyrene, a polyester, a polyether, a polyolefin, a polyalkylene oxide, a polyamide, a polyacrylate, a polymethacrylate and a polyurethane, or a mixture thereof. In a class of this embodiment, the microsphere is a polystyrene.

In certain embodiments, the microsphere contains carboxyl groups. In certain embodiments, the microsphere is coupled to a linker molecule prior to reacting the microsphere with the activated polysaccharide. In one embodiment, the linker compound is selected from the group consisting of α,ω-diaminoalkane and α,ω-alkanedihydrazide. In a class of this embodiment, the linker compound is adipic acid dihydrazide.

Another aspect of the present invention provides a method for assaying for an anti-polysaccharide antibody comprising contacting a sample containing said anti-polysaccharide antibody with a microsphere-polysaccharide conjugate prepared by the methods described herein; and measuring the amount of any anti-polysaccharide antibody bound to said microsphere-polysaccharide conjugate.

Another aspect of the present invention provides a method for detecting a disease, disorder or condition where anti-polysaccharide antibody levels are altered comprising the steps of: (a) contacting a sample of bodily tissue or fluid with a microsphere-polysaccharide conjugate prepared by the methods of the present invention, wherein said anti-polysaccharide antibody binds to said microsphere-polysaccharide conjugate; and (b) measuring the amount of any anti-polysaccharide antibody bound to said microsphere-polysaccharide conjugate, wherein the amount of said anti-polysaccharide antibody is diagnostic for said disease, disorder or condition.

Another aspect of the present invention provides a method for assessing the efficacy of a vaccine which vaccine alters anti-polysaccharide antibody levels in a mammal comprising the steps of: (a) administering an effective amount of said vaccine to said mammal; (b) allowing said mammal to develop anti-polysaccharide antibodies; (c) contacting a sample of bodily tissue or fluid from said mammal with a microsphere-polysaccharide conjugate prepared by the methods described herein, wherein said anti-polysaccharide antibody binds to said microsphere-polysaccharide conjugate; and (d) measuring the amount of any anti-polysaccharide antibody bound to said microsphere-polysaccharide conjugate, wherein the amount of said anti-polysaccharide antibody is diagnostic for the efficacy of said vaccine.

Another aspect of the present invention provides a biomolecule polysaccharide conjugate for the purpose of preparing vaccines synthesized by methods described herein.

Another aspect of the present invention provides a microsphere-polysaccharide conjugate synthesized by methods described herein.

Polysaccharides used in conjugates of the present invention may be of any kind. In one embodiment of the invention, the appropriate polysaccharides include capsular polysaccharides, polysaccharides derived from lipopolysaccharides (LPS) and lipooligosaccharides (LOS) of Gram-negative bacteria cell-wall, such as the O-specific side chain, and also fungal cell-wall polysaccharides. For example, polysaccharides may be isolated from bacteria including Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcusfaecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, Hemophilus influenza, Escherichia coli, Shigella spp., Erlichia spp., and Rickettsia spp., and from fungi such as Candida albicans, Candida kefyr, Cryptococcus neoformans, Hansenula anomala, and Hansenula arabitolgens. Polysaccharides isolated from the same bacteria may be also of different serotypes. For example, pneumococcal polysaccharides may be of the serotypes 1, 2, 3, 4, 5, 6B, 7F, 8, 9N, 9V, 10A, 11A, 12F, 14, 15B, 17F, 18C, 19A, 19F, 20, 22F, 23F, and 33F, and others.

Polysaccharides are composed of repeat units. For use in conjugates of the invention, in certain embodiments a polysaccharide comprises at least about 4 repeat units preferably up to about 3,000. Thus, the number-average degree of polymerization (the average number of glycose rings contained in one molecule) of the polysaccharide is at least about 4, with no particular upper limit, though it is preferably at most about 3,000. Especially for use as in an immunoassay, the number-average degree of polymerization of a polysaccharide is between about 4 and about 1,000, and particularly between about 4 and about 700, and more particularly between about 50 and about 200.

A repeat unit is characteristic of a given polysaccharide and thus the composition and molecular weight of the repeat unit greatly vary from one polysaccharide to another. For example, while the repeat unit of most capsular polysaccharides contains hydroxyl, carboxyl, and/or phosphoryl groups, some polysaccharides also contain amino groups (e.g. Streptococcus pneumoniae serotype 1), whereas others do not (e.g. Streptococcus pneumoniae serotype 14) and some polysaccharides contain N-acetyls (e.g. Streptococcus pneumoniae serotype 14), whereas others do not (e.g. Streptococcus pneumoniae serotype 6B). Also as a matter of example, the molecular weight of capsular polysaccharides of Streptococcus pneumoniae types 3 and 4 is 360 and 847, respectively. Thus, there is no general correspondence between the amount of repeat units and the molecular weight of the polysaccharide that may be globally applied, irrespective of the polysaccharide composition. However, one may independently indicate that a polysaccharide for use in the present invention has a preferred molecular weight in the average range of 1,000 to 2,500,000 daltons. The molecular weight of a polysaccharide is expressed as a mean value, since a polysaccharide is constituted by a population of molecules of heterogeneous size.

Polysaccharides may be either chemically synthesized, purified from a natural source according to conventional methods, or natural PSs can be further chemically modified. For example, in the case of bacterial or fungal polysaccharides, these latter may be extracted from the microorganisms and treated to remove the toxic moieties, if necessary. A particularly useful method is described by Gotschlich et al., J. Exp. Med., 129: 1349 (1969).

Polysaccharides may be used as synthesized or purified. They may be also depolymerized prior use. Indeed, native capsular polysaccharides usually have a molecular weight greater than 500,000 daltons. When it is preferred to use capsular polysaccharides of lower molecular weight, e.g. 10,000 to 20,000 daltons on average, polysaccharides as purified may be submitted to fragmentation. To this end, conventional methods are available. For example, WO 93/07178 describes a fragmentation method using an oxidation-reduction depolymerization reaction.

The term “polysaccharide” as used herein is meant to include compounds made up of many hundreds or even thousands of monosaccharide units per molecule. These units are held together by glycosidic linkages. Their molecular weights are normally greater than about 5,000 and can range up to millions of daltons. They are normally naturally-occurring, such as, for example, starch, glycogen, cellulose, gum arabic, agar, and chitin. The polysaccharide should have one or more reactive functional groups, such as hydroxyl, carboxyl, amino, phosphoryl, etc. The polysaccharide may be straight or branched chain.

The hydroxyl, carboxyl, phosphoryl, or amino groups of the polysaccharide that are involved in the linkage may be native functional groups. Alternatively, they may have been introduced artificially by chemical modification. Amino groups may be generated by controlled acidic or basic hydrolysis of native N-acyl groups such as N-acetyl groups. Hydrazide groups may be introduced by coupling the polymer with a linker, such as, e.g., adipic acid dihydrazide using conventional EDC-mediated coupling chemistry or other suitable means.

Polysaccharides that can be covalently linked according to methods described herein include starch-like and cellulosic material, but the present method is especially suitable for conjugating microbial polysaccharides that are haptens or immunogens. It is noted that the term “polysaccharides” as used herein comprises sugar-containing polymers and oligomers, whether they only contain glycosidic linkages or also phosphodiester or other linkages. They may also contain non-sugar moieties such as acid groups, phosphoryl groups, amino groups, hydroxyls and amino acids, and are optionally depolymerized.

A review of bacterial polysaccharides of interest can be found in Lennart Kenne and Bengt Lindberg, “Bacterial polysaccharides” in The polysaccharides, Vol. 2, Ed. G. O. Aspinall, 1983, Academic Press, pp. 287-363.

Other “biomolecules” which can be conjugated to PSs include enzymes, enzyme substrates, enzyme inhibitors, hormones, antibiotics, antibodies, antigens, peptides, polypeptides, proteins, other polysaccharides, nucleic acids, nucleosides, nucleotides, polynucleotides, and the like.

As used herein, the terms “covalent” and “valence” refer to a chemical bond between two atoms in a molecule created by the sharing of electrons, usually in pairs, by the bonded atoms and may involve single bonds or multiple bonds. The term “covalent” does not include hydrophobic/hydrophilic interactions, hydrogen-bonding, and van der Waals interactions.

The term “non-covalent” refers to interactions between two or more molecules and/or by two or more parts of the same molecule which are not “covalent” in nature. Such “non-covalent” interactions include electrostatic interactions such as, hydrogen bonds, hydrophobic/hydrophilic interactions, salt bridges, and van der Waals interactions.

The term “coating” as used herein refers to non-covalent immobilization of polysaccharides on solid surfaces, e.g., through adsorption. The nature of passive adsorption predominantly involves multiple hydrophobic interactions between solid phase and the polysaccharide.

The terms “immunogen” and “immunogenic” refer to substances capable of producing or generating an immune response in an organism directed specifically against the polysaccharide. The terms “antigenic” and “antigenicity” refer to the capability of a polysaccharide to be specifically bound by an antibody to the polysaccharide.

The term “immunospecific” means that the antibodies corresponding to the polysaccharide antigens exhibit a substantially greater affinity for the PSs attached to solid supports and biomolecules compared to the affinity for other antigens. It is also generally desirable that the affinity of antibodies corresponding to the polysaccharide antigens toward PSs attached to solid supports and biomolecules is similar to that toward the corresponding unattached PSs.

4-(4,6-Dimethoxy[1,3,5]triazin-2-yl)-4-methyl-morpholinium chloride (DMTMM) has the following structural formula: 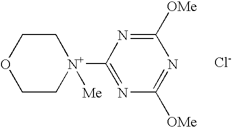
DMTMM is commercially available from the Sigma-Aldrich Chemical Company.

Microspheres, microparticles, microcapsules and beads, referred to herein collectively as “microspheres”, are solid or semi-solid particles having a diameter of less than one millimeter, more preferably less than 100 microns, which can be formed of a variety of materials, including synthetic polymers, proteins, and polysaccharides. Microspheres have been used in many different applications, primarily separations, diagnostics, and drug delivery. The most well-known examples of microspheres used in separations techniques are those which are formed of polymers of either synthetic or protein origin, such as polyacrylamide, hydroxyapatite or agarose. These polymeric microspheres are commonly used to separate molecules such as proteins based on molecular weight and/or ionic charge or by interaction with molecules chemically coupled to the microparticles. In the diagnostic area, microspheres are frequently used to immobilize an enzyme, substrate for an enzyme, or labeled antibody, which is then interacted with a molecule to be detected, either directly or indirectly. In the controlled drug delivery area, molecules are encapsulated within microparticles or incorporated into a monolithic matrix for subsequent release.

Microspheres have been commercially available as a tool for biochemists for many years. For example, antibodies conjugated to beads create relatively large particles specific for particular ligands. The large antibody-coated particles are routinely used to crosslink receptors on the surface of a cell for cellular activation, are bound to a solid phase for immunoaffinity purification, and may be used to deliver a therapeutic agent that is slowly released over time, using tissue or tumor-specific antibodies conjugated to the particles to target the agent to the desired site.

A number of different techniques are routinely used to make these microspheres from synthetic polymers, natural polymers, proteins and polysaccharides, including phase separation, solvent evaporation, emulsification, and spray drying. Examples of suitable polymers for the formation of microspheres include polystyrenes, polyesters, polyethers, polyolefins, polyalkylene oxides, polyamides, polyacrylates, polymethacrylates, polyurethanes, celluloses, polyisoprenes, silica, and polysaccharides, particularly cross-linked polysaccharides, such agarose, which is available as Sepharose, dextran, available as Sephadex and Sephacryl, cellulose, starch, and the like. Exemplary polymers used are addition polymers, such as polystyrene, polyvinyl alcohol, homopolymers and copolymers of derivatives of acrylate and methacrylate, particularly esters and amides having free hydroxyl functionalities. However, the availability and cost of these other polymeric particles make the use of polystyrene particles preferred. Other considerations which favor polystyrene are uniformity in the size and shape of the particles which are to be conjugated. The size of the polymer particles ranges from about 0.1 to about 100.0 μm. The preferred particle size is in the range of about 0.5 to 20.0 μm.

Other polymers used for the formation of microspheres include (a) homopolymers and copolymers of lactic acid and glycolic acid (PLGA) as described in U.S. Pat. No. 5,213,812 to Ruiz; U.S. Pat. No. 5, 417,986 to Reid et al.; U.S. Pat. No. 4,530,840 to Tice et al.; U.S. Pat. No. 4,897,268 to Tice et al.; U.S. Pat. No. 5,075,109 to Tice et al.; U.S. Pat. No. 5,102,872 to Singh et al.; U.S. Pat. No. 5,384,133 to Boyes et al.; U.S. Pat. No. 5,360,610 to Tice et al.; and European Patent Application Publication Number 248,531 to Southern Research Institute; (b) block copolymers such as tetronic 908 and poloxamer 407 as described in U.S. Pat. No. 4,904,479 to Illum; and (c) polyphosphazenes as described in U.S. Pat. No. 5,149,543 to Cohen et al.

Microspheres may be of a latex type. The term “latex,” as used herein, pertains to a stable colloidal dispersion of a polymeric substance in an aqueous medium. “Latex” is intended to mean an emulsion consisting substantially of latex mixed with water as a medium, but may also include additional ingredients such as bulking agents, fixing agents, adhesives, dyes and plasticizers, such latex compounds requiring heating to remove moisture and ensure effective adhesion. Also considered within the scope of the present invention are embodiments wherein the dispersion medium comprises an organic solvent. The dispersed particles preferably have an average particle size of about 0.1-100 μm, more preferably about 0.5-20 μm. The particle size distribution of the dispersed particles is not particularly limited, and the particles may have either wide particle size distribution or monodispersed particle size distribution. The polymer latex used in the present invention may be latex of the so-called core/shell type other than ordinary polymer latex having a uniform structure. In this case, use of different glass transition temperatures of core and shell may be preferred.

The naturally occurring or synthetic latex polymers are preferably derived from one or more unsaturated monomers which are capable of polymerizing in an aqueous environment. Particularly preferred are the use of any of the following monomers: (meth)acrylic based acids and esters, acrylonitrile, styrene, divinylbenzene, vinyl esters including but not limited to vinyl acetate, acrylamide, methacrylamide, vinylidene chloride, butadiene and vinyl chloride. The polymers that are produced may take the form of homopolymers (i.e., only one type of monomer selected) or copolymers (i.e., mixtures of two or more types of monomer are selected; this specifically includes terpolymers and polymers derived from four or more monomers). In one form, the copolymer could be a random, a block, or an alternating copolymer. Crosslinking is useful in many polymers for imparting structural integrity and rigidity to the microparticle.

Latex microspheres can be based on a range of synthetic polymers, such as polystyrene, polyvinyltoluene, polystyrene-acrylic acid, polyacrolein, and poly(meth)acrylate esters and their copolymers. The monomers used are normally water-insoluble, and are emulsified in aqueous surfactant so that monomer droplets and/or micelles are formed, which are then induced to polymerize by the addition of initiator to the emulsion. Substantially spherical monodisperse polymer particles are produced. By controlling the conditions, a variety of size ranges can be provided.

Microspheres are generally formed of a polymeric material that bears certain characteristics that make it useful in immunoassays. One such characteristic is that the matrix be inert to the components of the biological sample and to the assay reagents other than the assay reagent that is affixed to the microparticle. Other characteristics are that the matrix be solid and insoluble in the sample and in any other solvents or carriers used in the assay, and that it be capable of affixing an assay reagent to the microparticle. In certain preferred embodiments, these particles are functionalized by attaching a variety of chemical functional groups to their surfaces.

The surface of the solid phase will preferably contain functional groups for attachment of the polysaccharide. These functional groups can be incorporated into the polymer structure by conventional means, such as the forming the polymer from monomers that contain the functional groups, either as the sole monomer or as a co-monomer. Examples of suitable functional groups are amine groups (—NH₂), hydroxyl groups (—OH), phosphoryl groups (—O—P(O)(OH)₂) and carboxylic acid groups (—COOH). Useful monomers for introducing carboxylic acid groups into polystyrenes, for example, are acrylic acid and methacrylic acid.

Linking groups can be used as a means of increasing the density of reactive groups on the solid phase surface and decreasing steric hindrance to achieve maximal range and sensitivity for the assay, or as a means of adding specific types of reactive groups to the solid phase surface to broaden the range of types of assay reagents that can be affixed to the solid phase. Examples of suitable useful linking groups are adipic acid dihydrazide, polylysine, polyaspartic acid, polyglutamic acid and polyarginine.

In embodiments in which microspheres are used as the solid phase and detection is performed by flow cytometry, care should be taken to avoid the use of particles that emit high autofluorescence since this renders them unsuitable for flow cytometry. Microspheres of low autofluorescence can be created by standard emulsion polymerization techniques from a wide variety of starting monomers. Microspheres of high porosity and surface area (i.e., “macroporous” particles) as well as particles with a high percentage of divinylbenzene monomer should be avoided since they tend to exhibit high autofluorescence. Generally, however, microparticles suitable for use in this invention can vary widely in size, and the sizes are not critical to this invention. In most cases, best results will be obtained with microparticle populations whose particles range from about 0.1 μm to about 100 μm, preferably from about 0.5 μm to about 20 μm, in diameter.

Many such microspheres for use in conjugates and methods of the present invention are commercially available. They may be purchased from Luminex Corporation (Austin, Tex.) (xMAP™). xMAP™ microspheres are 5.6 μm in diameter and composed of polystyrene, divinylbenzene and methacrylic acid, which provides surface carboxylate functionality for covalent attachment of polysaccharides and biomolecules. The microspheres may be dyed with red- and/or infrared-emitting fluorochromes. By proportioning the concentrations of each fluorochrome, spectrally addressable microsphere sets may be obtained. When the microsphere sets are mixed and analyzed using the Luminex100™ instrument (Luminex), each set can be identified and classified by a distinct fluorescence signature pattern.

When particles are used as the solid phase, one means of separating bound from unbound species is to use particles that are made of or that include a magnetically responsive material. Such a material is one that responds to a magnetic field. Magnetically responsive materials that can be used in the practice of this invention include paramagnetic materials, ferromagnetic materials, ferrimagnetic materials, and metamagnetic materials. Paramagnetic materials are preferred. Examples are iron, nickel, and cobalt, as well as metal oxides such as Fe₃O₄, BaFel₂O₁₉, CoO, NiO, Mn₂O₃, Cr₂O₃, and CoMnP. The magnetically responsive material may constitute the entire particle, but is preferably only one component of the particle, the remainder being a polymeric material to which the magnetically responsive material is affixed and which is chemically derivatized as described above to permit attachment of an analyte binding member.

When particles containing magnetically responsive material are used, the quantity of such material in the particle is not critical and can vary over a wide range. The quantity can affect the density of the particle, however, and both the quantity and the particle size can affect the ease of maintaining the particle in suspension. Maintaining suspension serves to promote maximal contact between the liquid and solid phase and to facilitate flow cytometry. In assays where fluorescence plays a role in the detection, an excessive quantity of magnetically responsive material in the particles will also produce autofluorescence at a level high enough to interfere with the assay results. It is therefore preferred that the concentration of magnetically responsive material be low enough to minimize any autofluorescence emanating from the material. With these considerations in mind, the magnetically responsive material in a particle in accordance with this invention preferably ranges from about 1% to about 75% by weight of the particle as a whole. A more preferred weight percent range is from about 2% to about 50%, a still more preferred weight percent range is from about 3% to about 25%, and an even more preferred weight percent range is from about 5% to about 15%. The magnetically responsive material can be dispersed throughout the polymer, applied as a coating on the polymer surface or as one of two or more coatings on the surface, or incorporated or affixed in any other manner that secures the material in the polymer matrix.

While not wishing to be bound by theory, it is believed that the DMTMM-mediated formation of a microsphere-polysaccharide conjugate occurs as illustrated below. In one embodiment, the covalent conjugation of a polysaccharide to a microsphere begins in Step 1 (referring to the scheme below) with exposure of a carboxyl-containing polysaccharide to DMTMM which results in formation of a 4,6-dimethoxy-[1,3,5]triazin-2-yl ester of the polysaccharide. In a class of this embodiment, DMTMM is used in amounts ranging from substoichiometric to superstoichiometric with respect to the amount of reactive moieties present in the polysaccharide. In a subclass of this embodiment, DMTMM is used in a superstoichiometric amount with respect to the amount of reactive moieties present in the polysaccharide. The 4,6-dimethoxy-[1,3,5]triazin-2-yl ester of the polysaccharide is then reacted (Step 2) with a microsphere containing reactive moieties (A₁) resulting in the formation of the microsphere-polysaccharide conjugate. In one embodiment, the reactive moiety (A₁) is selected from the group consisting of hydroxyl, amino, carboxyl, and phosphoryl. In a class of this embodiment, the reactive moiety is carboxyl.

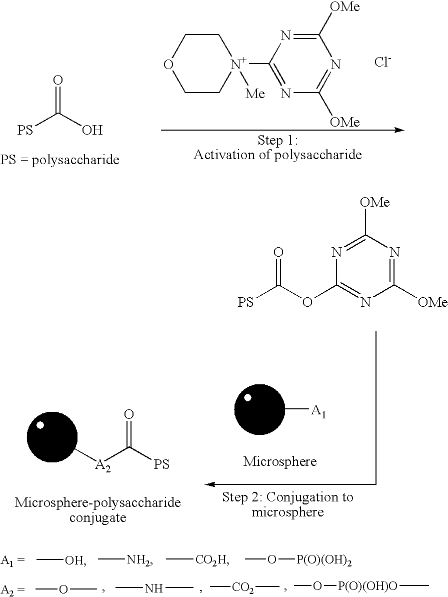

When the microsphere contains a carboxyl functionality, a carboxylic acid anhydride linkage is formed between the microsphere and the polysaccharide. When the microsphere contains a hydroxyl functionality, an ester linkage is formed between the microsphere and the polysaccharide. When the microsphere contains an amino functionality, an amide linkage is formed between the microsphere and the polysaccharide. When the microsphere contains a phosphoryl functionality, a mixed phosphoric acid carboxylic acid anhydride is formed between microsphere and the polysaccharide. In certain embodiments, a microsphere contains more than one type of reactive moieties and therefore the resultant microsphere-polysaccharide conjugate contains more than one type of linkage.

In another embodiment, the covalent conjugation of a microsphere to a polysaccharide begins in Step 1 with exposure of a carboxyl-containing microsphere to DMTMM which results in formation of a 4,6-dimethoxy-[1,3,5]triazin-2-yl ester of the microsphere. In a class of this embodiment, DMTMM is used in amounts ranging from substoichiometric to superstoichiometric with respect to the amount of reactive functionalities present on the microsphere. In a subclass of this embodiment, DMTMM is used in a superstoichiometric amount with respect to the amount of reactive moieties present on the microsphere. The 4,6-dimethoxy-[1,3,5]triazin-2-yl ester of the microsphere is then reacted (Step 2) with a polysaccharide containing a reactive functonality (A₁) resulting in the formation of the microsphere-polysaccharide conjugate. In one embodiment, the reactive functionality (A₁) is selected from the group consisting of hydroxyl, amino, carboxyl, and phosphoryl. In a class of this embodiment, the reactive moiety is carboxyl.

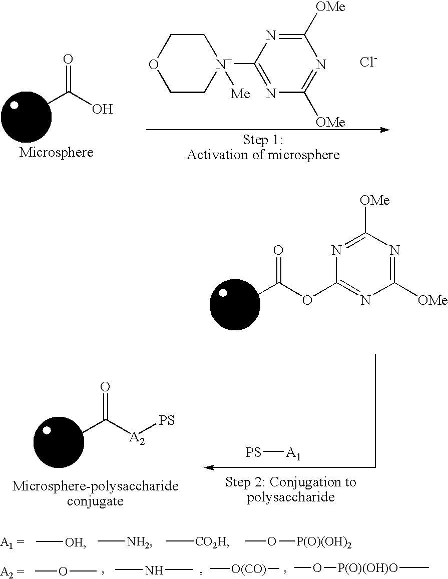

When the polysaccharide contains a carboxyl functionality, a carboxylic acid anhydride linkage is formed between the polysaccharide and the microsphere. When the polysaccharide contains a hydroxyl functionality, an ester linkage is formed between the polysaccharide and the microsphere. When the polysaccharide contains an amino functionality, an amide linkage is formed between the polysaccharide and the microsphere. When the polysaccharide contains a phosphoryl functionality, a mixed phosphoric acid carboxylic acid anhydride is formed between the polysaccharide and the microsphere. In certain embodiments, a polysaccharide contains more than one type of reactive moieties and therefore the resultant microsphere-polysaccharide conjugate contains more than one type of linkage.

The solvent may be any of the common solvents for chemical reactions unless the intended reaction is adversely affected. Examples of a solvent available for use in the methods of the present invention include protic solvents, such as water, phosphate buffered saline (PBS) and alcohol solvents (e.g., methanol, ethanol, n-propanol, isopropanol); and aprotic solvents, such as ethereal solvents (e.g., diethyl ether, tetrahydrofuran, dioxane, 1,2-dimethoxyethane), hydrocarbon solvents (e.g., benzene, toluene, hexane, heptane), halogenated hydrocarbon solvents (e.g, chloroform, dichloromethane, ethylene chloride) and other solvents including, e.g., acetone, acetonitrile, ethyl acetate, and N,N-dimethylformamide. Particularly, the solvent is water or PBS. These solvents may be used alone or, if necessary, in combination at an appropriate mixing ratio.

In one embodiment, reactions used for the covalent conjugation of a microsphere to a polysaccharide are carried out at temperatures ranging from −78° C. to 200° C. In a class of this embodiment reactions are carried out at −10° C. to 40° C. In a subclass of this class, reactions are carried at 10° C. to 25° C. In another subclass of this class, reactions are carried out at room temperature.

Referring to the schemes above, the intermediates obtained after Step 1, i.e., 4,6-dimethoxy-[1,3,5]triazin-2-yl esters of the polysaccharide and 4,6-dimethoxy-[1,3,5]triazin-2-yl esters of the microsphere may be purified prior to Step 2, or alternatively both steps (i.e., Step 1 and 2) may be performed in situ without purification of the intermediates. 4,6-Dimethoxy-[1,3,5]triazin-2-yl esters of the polysaccharide may be purified by standard chromatographic methods, e.g, by gel filtration on PD 10 columns (Amersham Biosciences, Piscataway, N.J.). 4,6-Dimethoxy-[1,3,5]triazin-2-yl esters of the microspheres may be separated by centrifugation or other conventional methods well known in the art. If particles containing a magnetically responsive material are used as the solid phase, separation may be achieved by placing the particles in a magnetic field, causing the particles to adhere to the walls of the reaction vessel. The particles once separated are washed to remove any remaining reagents (e.g., DMTMM). The particles can then be resuspended in a carrier liquid.

Microsphere-polysaccharide conjugates of the present invention may be used in a variety of immunoassays. Immunoassays of both the competitive type and the antibody-capture can be used. Competitive assays, for example, can be performed by using microspheres to which polysaccharides specific for the analyte are covalently bound. During the assay, the sample and a quantity of labeled analyte, either simultaneously or sequentially, are contacted with the microsphere-polysaccharide conjugates. By using a limited number of binding sites (polysaccharides) on the solid phase, the assay causes competition between the labeled analyte and the analyte in the sample for the available binding sites (polysaccharides). After a suitable incubation period, the mixture of liquid and solid is separated. If particles containing a magnetically responsive material are used as the solid phase, separation is achieved by placing the particles in a magnetic field, causing the particles to adhere to the walls of the reaction vessel. Otherwise, separation can be achieved by centrifugation or other conventional methods well known among those skilled in the use and design of immunoassays. The particles once separated are washed to remove any remaining unbound analyte and label. The particles can then be resuspended in a carrier liquid for introduction into, e.g., a flow cytometer where the label is detected.

Antibody capture assays, also known as solid-phase assays, are performed by using microspheres (or any solid phase) to which polysaccharides specific for the analyte are covalently bound. The bound polysaccharides are termed “capture” polysaccharides. An excess of capture polysaccharides is used relative to the suspected quantity range of the analyte so that all of the analyte binds. The solid phase with capture polysaccharides attached is placed in contact with the sample, and a second antigen to same analyte is added, simultaneously or sequentially with the sample. As with the capture polysaccharide, the second antigen is in excess relative to the analyte, but unlike the capture polysaccharide, the second antigen is conjugated to a detectable label, and may hence be referred to as “label” antigen. The capture polysaccharide and label antigen bind to different epitopes on the analyte or are otherwise capable of binding to the analyte simultaneously in a non-interfering manner. After a suitable incubation period, solid and liquid phases are separated. In the case where the solid phase consists of magnetically responsive microparticles, the liquid mixture with microparticles suspended therein is placed under the influence of a magnetic field, causing the microparticles to adhere to the walls of the reaction vessel, and the liquid phase is removed. The microparticles, still adhering to the vessel wall, are then washed to remove excess label antigen that has not become bound to the immobilized analyte, and the microparticles are then resuspended in a carrier liquid for introduction into a flow cytometer where the amount of label attached to the particles through the intervening analyte is detected.

Immunoassays in the practice of this invention can involve the detection of either monoclonal antibodies or polyclonal antibodies. Suppliers of such antibodies include Biotrend, Cologne, Germany; Biogenesis Inc., Brentwood, N.H., USA; Affinity Biologics, distributed by U.S. Enzyme Research Laboratories; Calbiochem, San Diego, Calif., USA; The Binding Site, Inc., San Diego, Calif., USA; Biodesign International, Saco, Me., USA; Enzyme Research Laboratories, Inc., South Bend, Ind., USA; Fitzgerald Industries International Inc., Concord, Mass., USA; and Hematologics Inc., Seattle, Wash., USA. For example, the analyte antibody for pneumococcal polysaccharides may be a polyclonal anti-human IgG detection antibody or it may be a well-characterized monoclonal anti-human IgG Fc detection antibody (e.g., clone HP6043) with uniform IgG isotype specificity.

Detection of the analyte in the practice of this invention can be accomplished by any of the wide variety of detection methods that are used or known to be effective in immunological assays. Fluorescence is one example and is readily achieved by the use of fluorophore labels. The wide variety of fluorophores and methods of using them in immunoassays are well known to those skilled in the immunoassay art, and a wide variety of fluorophores are commercially available. The preferred fluorophores are those that contribute as little autofluorescence as possible. The fluorophore phycoerythrin is preferred in this regard, since its extinction coefficient and quantum yield are superior to those of other fluorophores.

For embodiments of the invention that entail the use of flow cytometry, methods of and instrumentation for flow cytometry are known in the art. Examples of descriptions of flow cytometry instrumentation and methods in the literature are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); Steinkamp et al., Review of Scientific Instruments 44(9): 1301-1310 (1973); and Chandler, V. S., et al., U.S. Pat. No. 5,981,180 “Multiplexed Analysis of Clinical Specimens Apparatus and Methods,” issued Nov. 9, 1999 (Luminex Corporation).

The methods of this invention can be used in conjunction with any analytical procedures that are to be performed on serum or plasma samples for analytes indicative of a wide variety of physiological and clinical conditions. The two analyses can be performed either simultaneously or sequentially.

As used herein, the term “stoichiometric” means that the molar amount of DMTMM used is equal to the molar amount of reactive (towards DMTMM) moieties present in the polysaccharide or on a microsphere.

As used herein, the term “substoichiometric” means that the molar amount of DMTMM used is lower than the molar amount of reactive (towards DMTMM) moieties present in the polysaccharide or on a microsphere. In certain embodiments, substoichiometric amounts range from 0.025:1 (moles of DMTMM: moles of reactive moieties) up to a stoichiometric amount, particularly 0.25:1 up to a stoichiometric amount.

As used herein, the term “superstoichiometric” means that the molar amount of DMTMM used is greater than the molar amount of reactive (towards DMTMM) moieties present in the polysaccharide or on a microsphere. In certain embodiments, superstoichiometric amounts range up to 50:1 (moles of DMTMM: moles of reactive moieties), particularly 20:1, and more particularly 10:1.

The term “α,ω-diaminoalkane” as used herein is intended to include compounds of the formula H₂N-alkane-NH₂. The term “α,ω-alkanedihydrazide”, as used herein, is intended to include compounds of the formula H₂N—NH—C(O)-alkane-C(O)—NH—NH₂.

The term “enzyme immunoassay” includes any immunoassay in which an enzyme is part of the detection system. The enzyme may be simply a tag for an active component in the reaction mixture, or it may be assembled, disassembled, activated, or deactivated in the course of the reaction. The presence of the analyte of interest in the sample may be directly or inversely correlated with enzyme activity.

An “analyte” is a substance of interest to be measured in a sample using a particular assay system. It may have any size, structure, or valence irrespective of components used in the assay system, unless otherwise specified or required.

Some abbreviations used herein are as follows: EDAC (or EDC) is 1-ethyl-3-[3-(dimethylamino)propyl]-carbodiimide HCl; AATp=2-acetamido-4-amino-2,4,6,-trideoxy-D-galactose; D-Sug=2-acetamido-2,6-dideoxy-D-xylo-hexos-4-ulose; L-PneNAc=2-acetamido-2,6-dideoxy-L-talose; PLL is poly-(1)-lysine; PnPS is a capsular polysaccharide from Streptococcus pneumoniae; PnPS n is a capsular polysaccharide of serotype n from Streptococcus pneumoniae, e.g., PnPS 14 is a capsular polysaccharide of serotype 14 from Streptococcus pneumoniae. 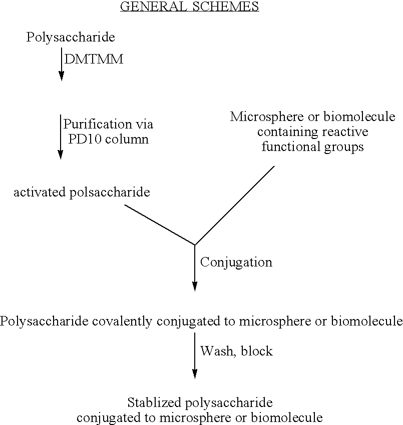
General Methodology to Activate and Covalently Conjugate Polysaccharides to Microspheres or Biomolecules:

Polysaccharides (2.5 mL at 1.0 mg/mL in distilled water) are activated with DMTMM (200 μL of 200 mg/mL distilled water) and incubated for 40 min on a rotator at room temperature. The entire volume (2.7 mL) is added to an equilibrated PD10 column (Amersham Biosciences, Piscataway, N.J.). The activated polysaccharide is eluted with 3.5 mL distilled water or PBS and added to microspheres or biomolecules containing a reactive functionality. After overnight incubation, the polysaccharides conjugated to the microspheres or biomolecules are washed, blocked and stored in a stabilizing buffer.

The following Examples are provided for purposes of illustration only and are not intended to limit the method of the present invention to the specific conditions for conducting the assay.

Pneumococcal Polysaccharides (PnPSs):

Twenty-three purified PnPS serotypes, namely serotypes 1, 2, 3, 4, 5, 6B, 7F, 8, 9N, 9V, 10A, 11A, 12F, 14, 15B, 17F, 18C, 19A, 19F, 20, 22F, 23F, and 33F, as well as purified PnPS serotypes 25 and 72 were obtained from Merck Manufacturing Division (MMD), (West Point, Pa.). The first 23 polysaccharides and polysaccharide PnPS 25 can be obtained from American Type Culture Collection (Rockville, Md.). Pneumococcal cell wall polysaccharide (C—PS) was obtained from Staten Seruminstitut (Copenhagen, Denmark). Stock aliquots of each PnPS serotype (1 mg/mL) and C-PS (1 mg/mL) were prepared in distilled water.

Serum Standard:

United States reference anti-pneumococcal serotype standard serum 89S-2 was obtained from C. Frasch, Center for Biologics Evaluation and Research, Food and Drug Administration (Bethesda, MD). This reference antiserum was prepared from 17 adult donors immunized with a 23-valent pneumococcal polysaccharide vaccine (PPV). A serum standard (identified in the Figures as NJSS) was prepared by combining two adult sera: one serum was from a paid adult volunteer vaccinated with PNEUMOVAX23® which was collected 30 days post vaccination in collaboration with G. Giebink, University of Minnesota; the second adult serum was obtained from a participant in a Merck Research Laboratories clinical trial. Both sera were obtained from individuals who consented to the use of their serum samples for reagents.

Luminex (xMAP™) Microspheres:

Microspheres (obtained from Luminex Corporation, Austin, Tex.) were 5.6 μm in diameter and composed of polystyrene, divinylbenzene and methacrylic acid, which provided surface carboxylate functionalities for covalent attachment of polysaccharides. Internally, the microspheres were dyed with red- and infrared-emitting fluorochromes. By proportioning the concentrations of each fluorochrome, spectrally addressable microsphere sets were obtained. When the microsphere sets were mixed and analyzed using the Luminex100™ instrument (Luminex, Austin, Tex.), each set was identified and classified by a distinct fluorescence signature pattern. In this study, several microsphere sets were used for covalent coupling of PnPSs and C—PS.

ADH-DMTMM Conjugation of Polysaccharides to Microspheres:

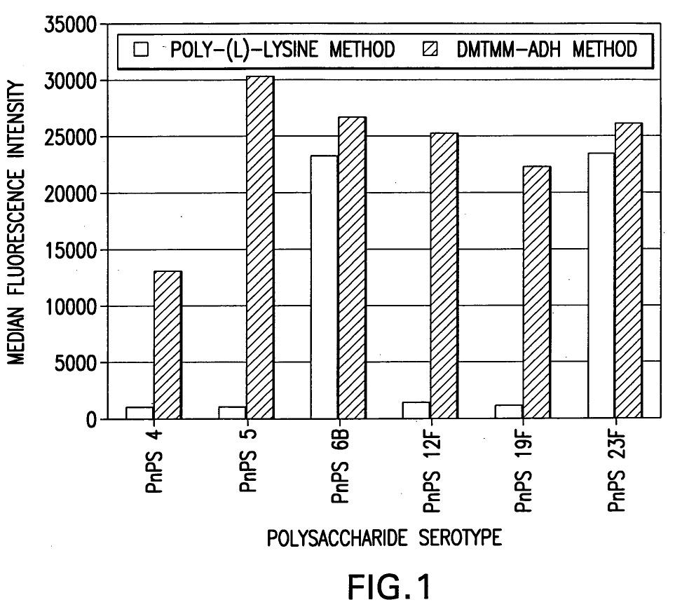

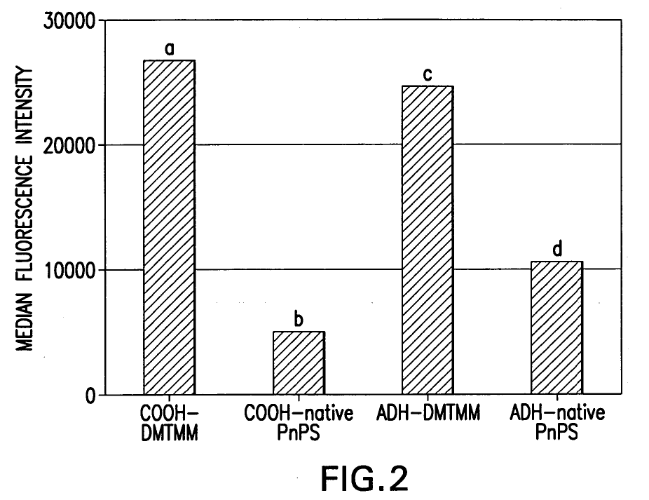

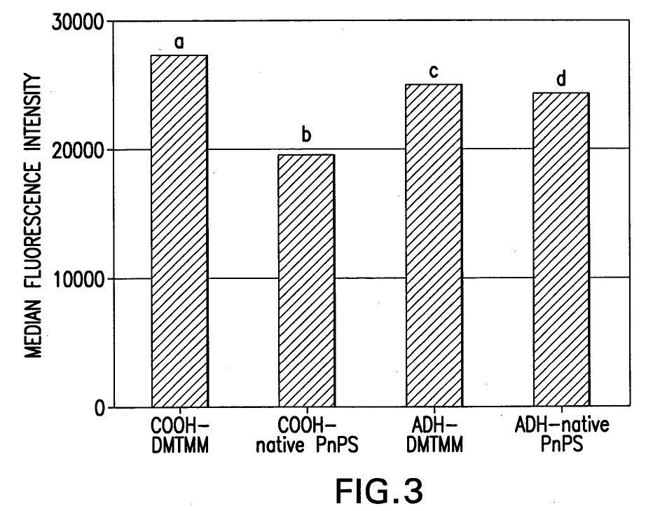

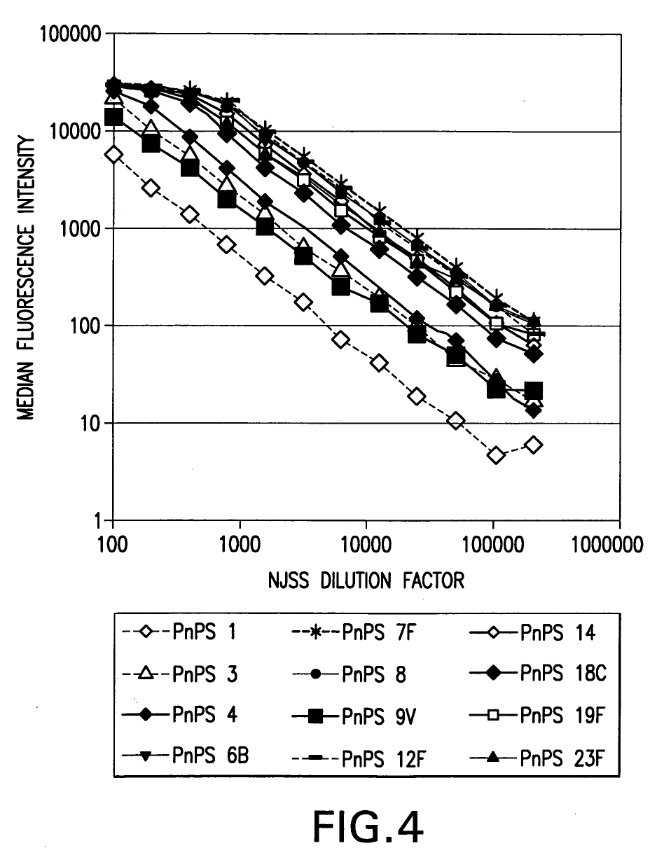

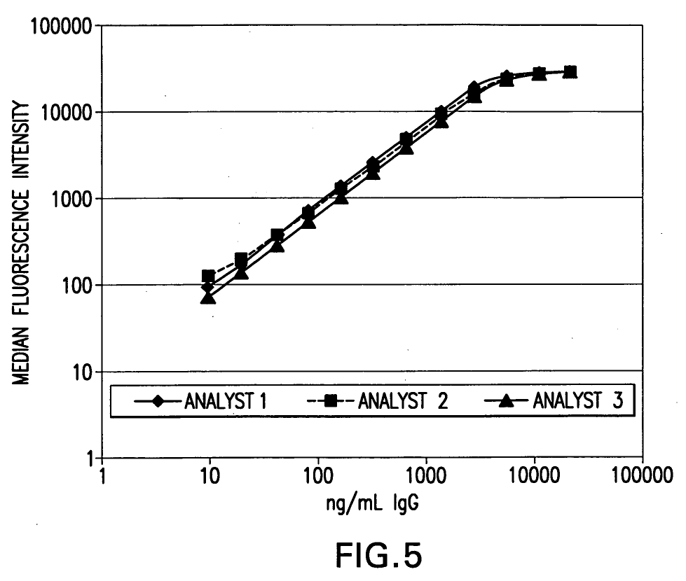

The carboxyl functional groups on microsphere surfaces were first modified using ADH (Aldrich, Milwaukee, Wis.). Into separate 1.5 mL microcentrifuge tubes (USA Scientific, Ocala, Fla.), 1.25×10⁷ microspheres from each microsphere set were added. The microspheres were washed by adding 500 μL of 100 mM 2-(N-morpholino)ethanesulfonic acid (MES), pH 6.0 (Sigma, St. Louis, Mo.), microcentrifuging at 13,200 rpm for 3-5 min at room temperature and aspirating the supernatant. The microsphere pellet was resuspended (all resuspensions were performed using sonication with a minisonicator [Cole Parmer, Vernon Hills, Ill.], and gentle vortexing [VWR, Intl., West Chester, Pa.]) in 1 mL of ADH (35 mg/mL, 100 mM MES, pH 6.0) and 200 μL of 200 mg/mL 1-ethyl-3-(3-dimethylaminopropyl)-carbodiimide hydrochloride) (EDC) (Pierce, Rockford, Ill.) in 100 mM MES, pH 6.0 (Sigma, St. Louis, Mo.). The tubes were rotated (Labquake; Barnstead/Thermolyne, Dubuque, Iowa) for 1 h at room temperature in the dark. The microspheres were then washed twice with 1 mL 100 mM MES, pH 4.5 (Sigma, St. Louis, Mo.) by microcentrifugation as described above, and the supernatants discarded. 500 μL of each DMTMM-activated polysaccharide eluent were added to the pellet of ADH-modified microspheres, and the microsphere/dialysate mixture incubated overnight at room temperature with rotation. The microspheres were again washed twice by microcentrifugation with PBS-0.05% Tween 20 (PBS-T) to remove any non-covalently bound polysaccharides and then treated with blocking buffer (10 mM PBS, 1% BSA, 0.05% NaN₃, Sigma, St. Louis, Mo.). The concentration of each PnPS-coupled microsphere set was determined using a hemacytometer and the microspheres were stored at 4° C. in the dark. To evaluate the new ADH-DMTMM conjugation chemistry, polysaccharide coupled microspheres using the ADH-DMTMM chemistry was compared against PnPS coupled microspheres using the poly-(1)-lysine chemistry in an Luminex immunoassay (described below). The PnPS microspheres when analyzed using a Luminex100™ instrument show improved signals using the ADH-DMTMM chemistry for several PnPS serotypes (FIG. 1).

COOH-DMTMM Conjugation of Polysaccharides to Microspheres:

For the covalent conjugations using the COOH-DMTMM method, in separate 5 mL vials (Coming Life Sciences, Acton, Mass.), 2.5 mL of a 1 μg/mL solution of each PnPS serotype and C-PS was treated with 200 μL of 200 mg/mL solution of 4-(4,6-dimethoxy[1,3,5]triazin-2-yl)-4-methyl-morpholinium chloride (DMTMM) in distilled water. The polysaccharide/DMTMM mixture was allowed to incubate 40-60 min on a rotator (Labquake; Barnstead/Thermolyne, Dubuque, Iowa) at room temperature. Following incubation, the mixture was added to equilibrated PD10 columns (Amersham, Piscataway, N.J.) and eluted with 3.5 mL PBS to separate out activated polysaccharides from free DMTMM. To prepare microspheres for conjugation, 12.5 million carboxylated microspheres (Luminex Corporation, Austin, Tex.) were dispensed into 1.5 mL centrifuge tubes (USA Scientific, Ocala, Fla.), and pelleted by microcentrifugation at 13,200 rpm for 3-5 min. Supernatant was removed and 500 μL of activated polysaccharide eluent was added. The microsphere-activated polysaccharide mixtures were vortexed [VWR, Intl., West Chester, Pa.] and sonicated with a minisonicator [Cole Parmer, Vernon Hills, Ill.], and incubated on a covered rotator overnight at room temperature. The microspheres were washed twice by microcentrifugation with PBS-Tween (PBS-T, 10 mM PBS, 0.05% Tween 20, pH 7.4, Merck Research Laboratories, West Point, Pa.) to remove any non-covalently bound polysaccharides and then treated with blocking buffer (10 mM PBS, 1% BSA, 0.05% NaN₃, Sigma, St. Louis, Mo.). The concentration of each PnPS-coupled microsphere set was determined using a hemacytometer and the microspheres were stored at 4° C. in the dark. Immunoassays (performed as described below) where carboxyl or ADH-modified microspheres were incubated with DMTMM activated or unactivated polysaccharides showed that the COOH-DMTMM chemistry yielded better results than the ADH-DMTMM chemistry for serotypes 14 (FIG. 2) and 18C (FIG. 3). However, both methods can produce good conjugation results.

PnPS Assay:

The steps in the assay were as follows. Fifty μL of the multiplexed microsphere mixture (where each set was at a concentration of approximately 1×10⁵microspheres/mL) were added to the wells of a 1.2 μm filter membrane microtiter plate (Millipore Corp. Part #MABVN1250, Bedford, Mass.) and liquid aspirated by use of a vacuum manifold filtration system (Millipore Part #MAVM09601). Standards prepared from serum 89S-2 or NJSS were diluted in two-fold dilutions beginning at 1:100 in PNEUMOVAX®diluent (PBS-1% BSA-0.05% Tween 20 diluent containing 10 μg/mL of C—PS, and 100 μg/mL of PNPS 25 and PnPS 72.) The diluted serum or diluent controls were added to the microsphere mixture in the wells of a 1.2 μm filter membrane microtiter plate (Millipore Corp. Part #MABVN1250, Bedford, Mass.) for 60 min at 37° C., with shaking. The liquid was then aspirated by use of a vacuum manifold filtration system (Millipore Part #MAVM09601). The microspheres were then washed 3 times with 200 μL PBS-T, each wash followed by vacuum aspiration. Fifty μL of 2 μg/mL (in blocking buffer) R-phycoerythrin conjugated mouse anti-human IgG (Clone HP6043, IgG2b; BIOTREND Intl., Destin, Fla.) were added to the wells of the plate and incubated for 30 min at 37° C. with shaking. After another PBS-T wash, the microspheres were resuspended in 125 μL PBS-T. The Luminex100™ instrument was programmed to inject 50 μL of the sample volume into the sample port at a rate of 60 μL/min to collect a minimum of 50 microspheres per set. An accessory X—Y (Luminex XYP) plate sampler was utilized to allow automated data collection and analysis directly from the 96-well plate. Acquisition software (Bio-Plex Manager 3.0, Bio-Rad Laboratories, Hercules, Calif.) was used to collect data. Using this assay, standard curves for each PnPS serotype can be generated (FIG. 4).

Data Analyses and Results:

To demonstrate coupling robustness, 14 different PnPS (1, 3, 4, 6B, 7F, 8, 9V, 12F, 14, 18C, 19F, 23F, 25, and 72) and C—PS microsphere were coupled to 15 different microspheres using the COOH-DMTMM procedures above by three separate individuals. A five parameter logistic (5-PL) model within the Bio-Plex Manager 3.0 software was used to fit the relationship between median fluorescence intensity (MFI) and anti-PnPS IgG concentrations (Bio-Rad Laboratories, Hercules, Calif.). The standard curves produced using the NJSS standard produced similar results for all three individuals, suggesting this coupling method is reproducible (See FIG. 5). To assess specificity and to verify antigenicity of each coupled polysaccharide, each PnPS was added singly as a competitor to different wells containing the multiplexed microsphere mix and serum NJSS or 89s-2 added at 1:100 dilution in PNEUMOVAX® diluent. Specifically, a final concentration of 100 μg/mL solution of each PnPS was used for overnight pre-incubation. Following overnight incubation, the assay was performed as specified above using either 50 μL of the serum diluted in PNEUMOVAX® diluent containing the specific inhibitor or as a control, 50 μL of the serum diluted in PNEUMOVAX® diluent alone without additional inhibitors. Specificity results for each microsphere-polysaccharide using homologous and heterologous inhibition were determined by calculating the percent inhibition in MFI signal in the presence of the polysaccharide inhibitor relative to the MFI signal without the added inhibitor (see Table 1 below): Percent inhibition=10% * [(MFI using PNEUMOVAX diluent alone)−(MFI using added inhibitor)]/(MFI using PNEUMOVAX diluent alone). The specificity results showed that antigenicity of the polysaccharides was not harmed by the coupling method.

Table 1: Specificity of multiplexed immunoassay using COOH-DMTMM method for preparing PnPS-microspheres shown as percent inhibition of signal due to competing free polysaccharides.

TABLE 1
Specificity of multiplexed immunoassay using COOH-DMTMM method for preparing PnPs-
microspheres shown as percent inhibition of signal due to competing free polysaccharides.

| | | Percent inhibition for PnPS-microsphere | | | | | | | | | | | |
|---|---|---|---|---|---|---|---|---|---|---|---|---|---|
| | | 1 | 3 | 4 | 6B | 7F | 8 | 9V | 12F | 14 | 18C | 19F | 23F |
| Added | 1 | 98% | −6% | −1% | −1% | 0% | 0% | −12% | 0% | 0% | −1% | 1% | 0% |
| inhibi- | 3 | −11% | 99% | −1% | 0% | 0% | −1% | −14% | −1% | −1% | 0% | −1% | 0% |
| tor | 4 | −9% | −1% | 99% | 0% | 1% | 0% | −2% | 0% | 0% | 0% | 0% | 0% |
| | 6B | 12% | −6% | 1% | 98% | −1% | 0% | 13% | −1% | −1% | −2% | −2% | −1% |
| | 7F | 7% | −4% | −1% | −1% | 99% | 0% | −4% | 0% | 0% | −1% | 0% | −1% |
| | 8 | −9% | −3% | −3% | −2% | 0% | 99% | −6% | −1% | −1% | −1% | 0% | −1% |
| | 9V | 6% | −1% | 3% | 1% | 3% | 2% | 99% | 4% | 2% | 2% | 3% | 1% |
| | 12F | 0% | −4% | 0% | −2% | 0% | 0% | −3% | 98% | −1% | −2% | −1% | −2% |
| | 14 | −7% | −1% | −3% | 0% | −1% | 0% | −6% | 0% | 99% | −1% | 0% | −1% |
| | 18C | 10% | −2% | −1% | 0% | 1% | 1% | −1% | 1% | 0% | 98% | 0% | 1% |
| | 19F | −5% | −2% | −2% | −1% | 0% | 0% | −8% | 0% | 0% | 0% | 99% | 0% |
| | 23F | −9% | −5% | −3% | 0% | 0% | 0% | −14% | −1% | 0% | 0% | 0% | 99% |

1. A method for coupling a polysaccharide to a microsphere or biomolecule comprising the steps of:
(a) activating said polysaccharide with 4-(4,6-dimethoxy[1,3,5]triazin-2-yl)-4-methyl-morpholinium chloride, and
(b) reacting said activated polysaccharide with said microsphere or biomolecule.

2. A method for coupling a polysaccharide to a microsphere or biomolecule comprising the steps of:
(a) activating said microsphere or biomolecule with 4-(4,6-dimethoxy[1,3,5]triazin-2-yl)-4-methyl-morpholinium chloride, and
(b) reacting said activated microsphere or biomolecule with said polysaccharide.

3. The method of claim 1 or 2 wherein said polysaccharide is a bacterial polysaccharide.

4. The method of claim 3 wherein said bacterial polysaccharide is isolated from a bacterium selected from the group consisting of Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcusfaecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, Hemophilus influenzae, Escherichia coli, Shigella spp., Erlichia spp., and Rickettsia spp.

5. The method of claim 4 wherein said bacterial polysaccharide is isolated from Streptococcus pneumoniae.

6. The method of claim 5 wherein said bacterial polysaccharide is a capsular polysaccharide.

7. The method of claim 6 wherein said capsular polysaccharide is of a serotype selected from the group consisting of: 1, 2, 3, 4, 5, 6B, 7F, 8, 9N, 9V, 10A, 11A, 12F, 14, 15B, 17F, 18C, 19A, 19F, 20, 22F, 23F, and 33F.

8. The method of claim 1 or 2 wherein said polysaccharide comprises about 4 to about 3000 repeat units.

9. The method of claim 8 wherein said polysaccharide comprises about 4 to about 1000 repeat units.

10. The method of claim 9 wherein said polysaccharide comprises about 4 to about 700 repeat units.

11. The method of claim 10 wherein said polysaccharide comprises about 50 to about 200 repeat units.

12. The method of claim 1 or 2 wherein said microsphere comprises a polymer selected from the group consisting of a polystyrene, a polyester, a polyether, a polyolefin, a polyalkylene oxide, a polyamide, a polyacrylate, a polymethacrylate and a polyurethane, or a mixture thereof.

13. The method of claim 12 wherein said polymer is a polystyrene.

14. The method of claim 1 or 2 wherein said microsphere or biomolecule is substituted with a reactive functionality selected from the group consisting of hydroxyl, amino, carboxyl, and phosphoryl.

15. The method of claim 1 or 2 wherein said polysaccharide is substituted with a reactive functionality selected from the group consisting of hydroxyl, amino, carboxyl, and phosphoryl.

16. The method of claim 14 wherein said reactive functionality is carboxyl.

17. The method of claim 15 wherein said reactive functionality is carboxyl.

18. The method of claim 1 or 2 wherein said polysaccharide contains one or more residues of glycuronic acid.

19. The method of claim 1 wherein said microsphere is coupled to a linker molecule prior to reacting said microsphere with said activated polysaccharide.

20. The method of claim 19 wherein said linker molecule is an α,ω-diaminoalkane or an α,ω-alkanedihydrazide.

21. The method of claim 20 wherein said α,ω-alkanedihydrazide is adipic acid dihydrazide.

22. A method for assaying for an anti-polysaccharide antibody comprising the steps of:
(a) contacting a sample containing said anti-polysaccharide antibody with a microsphere-polysaccharide conjugate prepared by the method of claim 1 or 2; and
(b) measuring the amount of anti-polysaccharide antibody bound to said microsphere-polysaccharide conjugate.

23. A method for detecting a disease, disorder or condition where anti-polysaccharide antibody levels are altered comprising the steps of:
(a) contacting a sample of bodily tissue or fluid with a microsphere-polysaccharide conjugate prepared by the method of claim 1 or 2, wherein said anti-polysaccharide antibody binds to said microsphere-polysaccharide conjugate; and
(b) measuring the amount of anti-polysaccharide antibody bound to said microsphere-polysaccharide conjugate, wherein the amount of said anti-polysaccharide antibody is diagnostic for said disease, disorder or condition.

24. A method for assessing the efficacy of a vaccine which vaccine alters anti-polysaccharide antibody levels in a mammal comprising the steps of:
(a) administering an effective amount of said vaccine to said mammal;
(b) allowing said mammal to develop anti-polysaccharide antibodies;
(c) contacting a sample of bodily tissue or fluid from said mammal with a microsphere-polysaccharide conjugate prepared by the method of claim 1 or 2, wherein said anti-polysaccharide antibody binds to said microsphere-polysaccharide conjugate; and
(d) measuring the amount of anti-polysaccharide antibody bound to said microsphere-polysaccharide conjugate, wherein the amount of said anti-polysaccharide antibody is diagnostic for the efficacy of said vaccine.

25. A microsphere-polysaccharide conjugate prepared by the process of claim 1 or 2.

26. A biomolecule-polysaccharide conjugate prepared by process of claim 1 or 2.
